SEQUENCE LISTING (18 sequences)

SEQ ID NO: 1 - protein, 15 aa, Artificial sequence
  Synthetic sequence Tropoelastin-specific binding peptide
VVGSPSAQDEASPLS

SEQ ID NO: 2 - protein, 6 aa, Artificial sequence
  Synthetic sequence Tropoelastin hexapeptide
VGVAPG

SEQ ID NO: 3 - protein, 11 aa, Artificial sequence
  Synthetic sequence Conjugate
KYPDHVQYTHY

SEQ ID NO: 4 - protein, 6 aa, Artificial sequence
  Synthetic sequence Tropoelastin-specific binding peptide
EGFEPG

SEQ ID NO: 5 - protein, 10 aa, Artificial sequence
  Synthetic sequence Tropoelastin-specific binding peptide
YPDHVQYTHY

SEQ ID NO: 6 - protein, 4 aa, Artificial sequence
  Synthetic sequence Tropoelastin-specific binding peptide
QDEA

SEQ ID NO: 7 - protein, 16 aa, Artificial sequence
  Synthetic sequence Conjugate
VVGSPSAQDEASPLSK

SEQ ID NO: 8 - protein, 17 aa, Artificial sequence
  Synthetic sequence Conjugate
KVVGSPSAQDEASPLSK

SEQ ID NO: 9 - protein, 17 aa, Artificial sequence
  Synthetic sequence Conjugate
KKVVGSPSAQDEASPLS

SEQ ID NO: 10 - protein, 16 aa, Artificial sequence
  Synthetic sequence Conjugate
KVVGSPSAQDEASPLS

SEQ ID NO: 11 - protein, 12 aa, Artificial sequence
  Synthetic sequence Conjugate
KYPDHVQYTHYK

SEQ ID NO: 12 - protein, 11 aa, Artificial sequence
  Synthetic sequence Conjugate
YPDHVQYTHYK

SEQ ID NO: 13 - protein, 12 aa, Artificial sequence
  Synthetic sequence Conjugate
KKYPDHVQYTHY

SEQ ID NO: 14 - protein, 786 aa, Homo sapiens
MAGLTAAAPRPGVLLLLLSILHPSRPGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGAAGAGVLGGLVPGPQAAVPGVPGTGGVPGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 15 - protein, 860 aa, Mus musculus
MAGLTAVVPQPGVLLILLLNLLHPAQPGGVPGAVPGGLPGGVPGGVYYPGAGIGGLGGGGGALGPGGKPPKPGAGLLGTFGAGPGGLGGAGPGAGLGAFPAGTFPGAGALVPGGAAGAAAAYKAAAKAGAGLGGVGGVPGGVGVGGVPGGVGVGGVPGGVGVGGVPGGVGGIGGIGGLGVSTGAVVPQVGAGIGAGGKPGKVPGVGLPGVYPGGVLPGTGARFPGVGVLPGVPTGTGVKAKAPGGGGAFSGIPGVGPFGGQQPGVPLGYPIKAPKLPGGYGLPYTNGKLPYGVAGAGGKAGYPTGTGVGSQAAAAAAKAAKYGAGGAGVLPGVGGGGIPGGAGAIPGIGGIAGAGTPAAAAAAKAAAKAAKYGAAGGLVPGGPGVRLPGAGIPGVGGIPGVGGIPGVGGPGIGGPGIVGGPGAVSPAAAAKAAAKAAKYGARGGVGIPTYGVGAGGFPGYGVGAGAGLGGASPAAAAAAAKAAKYGAGGAGALGGLVPGAVPGALPGAVPAVPGAGGVPGAGTPAAAAAAAAAKAAAKAGLGPGVGGVPGGVGVGGIPGGVGVGGVPGGVGPGGVTGIGAGPGGLGGAGSPAAAKSAAKAAAKAQYRAAAGLGAGVPGFGAGAGVPGFGAGAGVPGFGAGAGVPGFGAGAGVPGFGAGAVPGSLAASKAAKYGAAGGLGGPGGLGGPGGLGGPGGLGGAGVPGRVAGAAPPAAAAAAAKAAAKAAQYGLGGAGGLGAGGLGAGGLGAGGLGAGGLGAGGLGAGGLGAGGLGAGGGVSPAAAAKAAKYGAAGLGGVLGARPFPGGGVAARPGFGLSPIYPGGGAGGLGVGGKPPKPYGGALGALGYQGGGCFGKSCGRKRK

SEQ ID NO: 16 - protein, 417 aa, Homo sapiens
MRFAWTVLLLGPLQLCALVHCAPPAAGQQQPPREPPAAPGAWRQQIQWENNGQVFSLLSLGSQYQPQRRRDPGAAVPGAANASAQQPRTPILLIRDNRTAAARTRTAGSSGVTAGRPRPTARHWFQAGYSTSRAREAGASRAENQTAPGEVPALSNLRPPSRVDGMVGDDPYNPYKYSDDNPYYNYYDTYERPRPGGRYRPGYGTGYFQYGLPDLVADPYYIQASTYVQKMSMYNLRCAAEENCLASTAYRADVRDYDHRVLLRFPQRVKNQGTSDFLPSRPRYSWEWHSCHQHYHSMDEFSHYDLLDANTQRRVAEGHKASFCLEDTSCDYGYHRRFACTAHTQGLSPGCYDTYGADIDCQWIDITDVKPGNYILKVSVNPSYLVPESDYTNNVVRCDIRYTGHHAYASGCTISPY

SEQ ID NO: 17 - protein, 411 aa, Mus musculus
MRFAWAVLLLGPLQLCPLLRCAPQTPREPPAAPGAWRQTIQWENNGQVFSLLSLGAQYQPQRRRDPSATARRPDGDAASQPRTPILLLRDNRTASTRARTPSPSGVAAGRPRPAARHWFQAGFSPSGARDGASRRAANRTASPQPPQLSNLRPPSHIDRMVGDDPYNPYKYSDDNPYYNYYDTYERPRPGSRNRPGYGTGYFQYGLPDLVPDPYYIQASTYVQKMSMYNLRCAAEENCLASSAYRADVRDYDHRVLLRFPQRVKNQGTSDFLPSRPRYSWEWHSCHQHYHSMDEFSHYDLLDANTQRRVAEGHKASFCLEDTSCDYGYHRRFACTAHTQGLSPGCYDTYAADIDCQWIDITDVQPGNYILKVSVNPSYLVPESDYTNNVVRCDIRYTGHHAYASGCTISPY

SEQ ID NO: 18 - protein, 404 aa, Artificial sequence
  Synthetic sequence Predicted rabbit lysyl oxidase
MLCSWTVLLLGPLQLCALVCGAPQAAGQQQPPREPPAAPGAWRQRIQWENNGQVFSLLSLGAQYQPQRRRDAGAAAPGAQRAAGPQQRTPVLLLRDNRTAAASRPRPAGRHWFQAGYASPGARDAGASRAGNRTAQGEPPALSNLRPPSHVDRMVGDDPYNPYKYSDDNPYYNYYDTYERPRPGSRYRPGYGTGYFQYGLPDLVPDPYYIQASTYVQKMSMYNLRCAAEENCLASSAYRADVRDYDHRVLLRFPQRVKNQGTSDFLPSRPRYSWEWHSCHQHYHSMDEFSHYDLLDANTQRRVAEGHKASFCLEDTSCDYGYHRRFACTAHTQGLSPGCYDTYAADIDCQWIDITDVQPGNYILKVSVNPSYLVPESDYTNNVVRCDIRYTGHHAYASGCTISP

(19) United States
(12) Patent Application Publication
(10) Pub. No.: US 2014/0065069 A1
(43) Pub. Date: Mar. 6, 2014

(54) Materials and Methods Relating to Cardiovascular Imaging

(71) Applicants: Rene Botnar, London (GB); Alkystis Phinikaridou, London (GB)

(72) Inventors: Rene Botnar, London (GB); Alkystis Phinikaridou, London (GB)

(73) Assignee: KING'S COLLEGE LONDON, London (GB)

(21) Appl. No.: 13/984,522

(22) PCT Filed: Feb. 8, 2012
(86) PCT No.: PCT/GB12/00133

§ 371 Date: Nov. 22, 2013

(30) Foreign Application Priority Data
Feb. 8, 2011 (GB) 1102189.6

(51) Int. Cl. A61K 51/08

(52) U.S. Cl. CPC A61K 51/088
USPC 424/1.69; 530/327; 530/329; 530/328

(57) ABSTRACT

The invention provides conjugates for imaging plaques, such as cardiovascular plaques, as well as associated pharmaceutical compositions. Other aspects of the invention include methods for administering and imaging such conjugates and compositions, and using the imaging to characterise plaques. The conjugates of the invention distinguish between tropoelastin and elastin in plaques. The presence of tropoelastin can act as an indication that a plaque is liable to rupture or erode. Such information allows assessment of disease progression and response to treatment.

### FIELD OF THE INVENTION

The present invention relates to materials and methods relating to plaque imaging, and more particularly the imaging of cardiovascular plaques using agents that are capable of imaging plaques for assessing plaque burden and instability, disease progression and response to therapy.

### BACKGROUND OF THE INVENTION

Acute myocardial infarction (AMI) and stroke remain the leading causes of mortality and morbidity in Western countries. AMI is predominantly caused by the rupture or erosion of unstable/vulnerable atherosclerotic plaques. A complex group of biological processes are associated with plaque progression and destabilization including endothelial dysfunction, inflammation, neovascularization, outward remodelling and extracellular matrix disorganization. Similarly, aortic aneurysm development and rupture is thought to be the result of inflammation and matrix degradation.

The assessment of plaque burden and instability, progression of disease and the evaluation of response to therapies have been the subject of research in this area as it would be desirable to be able to distinguish plaques that are likely to rupture/erode.

Early studies using coronary-angiography, a technique limited to indirect visualization of the coronary vessel wall, have established a relationship between the extent of disease, disease progression and associated cardiovascular mortality. Intravascular ultrasound (IVUS) and optical-coherence-tomography were developed to image the vessel wall with high spatial resolution, enabling precise quantification of plaque burden. However, the invasive nature of these techniques precludes screening or follow-up investigations in large patient populations. Disease burden and progression have been established as independent predictors for adverse outcomes. FDG-PET has been shown to be associated with plaque macrophage content as well as with imaging features of vulnerable plaques including echolucency on IVUS, plaque haemorrhage and lipid rich plaque on MR as well as uptake of a macrophage-specific CT contrast agent.

Molecular magnetic resonance imaging (MRI) is a non-invasive technique, allowing the visualization of biological markers in vivo. As significantly higher spatial resolution can be achieved compared to other clinical molecular imaging modalities, it is well suited for the evaluation of the relatively thin arterial vessel wall. By way of example, WO 2007/05491 discloses the use of hydrazide conjugates as MRI agents for imaging plaques. However, even though progress has been made in the design of high relaxivity contrast agents, sensitivity remains a major limiting factor for molecular MRI compared to positron-emission-tomography, single-photon-emission-computed-tomography and optical imaging.

The presence of elastin and tropoelastin in arterial plaques has been the subject of research. Krettek et al. (1), describe the increase in tropoelastin in human atheroma and abdominal aortic aneurysms in comparison to non-diseased arteries. They also show that macrophages may be the source of the tropoelastin. Xu et al. (2) describe tropoelastin expression as closely associated with the development of foam cells lesions. Akima et al. (3) describe a high level of elastin mRNA, but low levels of elastin in lipid-rich and ruptured plaques.

Visualisation of tropoelastin and elastin has been approached in different ways; Kozel et al. (4) make use of an antibody labeled with dye to visualise elastin in cells, and Starcher et al. (5) describe antibodies to epitopes on tropoelastin, but not elastin. WO2011/005322 (6) describes compounds for imaging elastin rich tissues.

Other conjugates have been used to examine vascular injury. In U.S. Pat. No. 5,972,890 (7), it is suggested that peptide-labeled conjugates are used to bind to sites of vascular injury. U.S. Pat. No. 4,877,599 (8) describes the use of antibodies to human elastin conjugated to I-125, in rabbits.

Accordingly, there remains a need in the art to provide further methods for imaging plaques, and in particular for assessing whether plaques are liable to rupture/erode.

### SUMMARY OF THE INVENTION

Broadly, the present invention is based on the finding that vulnerable plaques at risk of rupture or erosion have increased tropoelastin content compared to stable plaques and that imaging agents that are capable of specifically binding to tropoelastin can be used for imaging plaques, for example for assessing plaque burden and instability, disease progression and/or response to therapy. Alternatively or additionally, the present invention includes the use of lysyl oxidase as a marker for unstable plaques based on results disclosed herein that show that lysyl oxidase activity is reduced in unstable plaques as compared to plaques that are stable. Without wishing to be bound by any particular theory, these findings are linked as lysyl oxidase is the enzyme responsible for cross-linking tropoelastin to produce mature elastin. Accordingly, the present invention provides a means for improving the detection of unstable rupture prone plaques using novel tropoelastin specific contrast agents and/or imaging agents for detecting the presence, amount or activity of lysyl oxidase, and thus allows better guiding treatment in this high-risk patient population.

Elastin plays an important structural role in the vessel wall, but also has biological signalling functions. Several pathological stimuli may be responsible for triggering elastogenesis in atherosclerosis leading to a marked increase in elastin content during plaque development. Immature elastic fibers may represent an atherogenic stimulus for the recruitment of proinflammatory cells. Imaging quantitative changes in intraplaque elastin content may yield complementary information for assessment of plaque burden alone, especially, as it was indicated that human atherosclerotic plaques could potentially be differentiated into fibrous and atheromatous subtypes, based on their relative elastin content.

Accordingly, in a first aspect, the present invention provides a conjugate for imaging plaques comprising a tropoelastin-specific binding agent or a lysyl oxidase-specific binding agent, wherein the binding agent is linked to an imaging probe.

In a further aspect, the present invention provides a conjugate for use in a method of imaging plaques comprising a tropoelastin-specific binding agent or a lysyl oxidase-specific binding agent, wherein the binding agent is linked to an imaging probe.

In a further aspect, the present invention provides the use of a conjugate in the preparation of a medicament for imaging plaques, wherein the conjugate comprises a tropoelastin-specific binding agent and an imaging probe.

The present invention may relate to the imaging of cardiovascular plaques. In some cases, in accordance with any one of the aspects of the present invention, the plaques may be cardiovascular plaques. In some cases, in accordance with any one of the aspects of the present invention, the plaques may be atherosclerotic cardiovascular plaques.

In a further aspect, the present invention provides a pharmaceutical composition comprising a conjugate of the invention. Typically, the compositions will be for intravenous administration to a patient.

In a further aspect, the present invention provides a method of imaging cardiovascular plaques in a subject, the method comprising:
(a) administering to the subject a composition comprising a conjugate for imaging cardiovascular plaques comprising a tropoelastin-specific binding agent and an imaging probe;
(b) allowing the imaging agent to bind to any tropoelastin present in plaques in the vascular system of the subject;
(c) detecting the imaging probe to determine the presence of the plaques.

Accordingly, the methods of the present invention may be used to determine the likelihood of a patient developing a condition caused by plaque rupture or instability by imaging of cardiovascular plaques, for example atherosclerotic plaques, with the conjugate, for example acute myocardial infarction (AMI), stroke and/or aortic aneurysm. Additionally or alternatively, the methods of the present invention may further comprise using the imaging of the cardiovascular plaques, for example atherosclerotic plaques, with the conjugate for (i) determining a course of treatment for a patient; and/or (ii) assigning a patient to a class of patients for a given therapy; and/or (iii) assessing plaque burden, (iv) monitoring disease progression and/or (v) determining the response of a patient to a therapy. As part of any of these methods, step (c) may comprise quantifying the tropoelastin present in plaques.

Embodiments of the present invention will now be described by way of example and not limitation with reference to the accompanying figures and examples.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments, which are described.

### BRIEF DESCRIPTION OF THE FIGURES

FIG. 1. Scheme showing the production of elastin from tropoelastin.

FIG. 2. Quantitation of tropoelastin fibers in stable and unstable rabbit plaque with IHC showing that there is upregulation of tropoelastin in unstable versus stable plaque.

FIG. 3. Quantitation of total elastin (tropoelastin and mature elastin) fibres in stable and vulnerable rabbit plaques showing that vulnerable plaques have increased total elastin (tropoelastin+mature elastin) content compared to stable plaques.

FIG. 4. LOX is down-regulated in vulnerable plaques.

FIG. 5. Illustration showing the peptide sequence VVGSPSAQDEASPLS binding the hexapeptide VGVAPG on tropoelastin.

FIG. 6. In vivo imaging of plaques in ApoE^{-/-} mouse model with gadolinium labeled (DOTA-Gd)-VVGSPSAQDEASPLS showing preferential uptake of the conjugate in plaque-laden brachiocephalic artery and aortic arch but no uptake in plaque-free carotid artery.

FIG. 7. In vivo imaging of in ApoE^{-/-} mouse model with gadolinium labelled K-(DOTA-Gd)-YPDHVQYTHY showing preferential uptake of the conjugate in plaques-laden brachiocephalic artery and aortic arch but no uptake in plaque-free carotid artery.

FIG. 8. Immunohistochemistry: Tropoelastin staining (brown) confirms presence of tropoelastin in the neointima (white arrow) and adventitia (black arrow) in the diseased brachiocephalic artery, but no to little tropoelastin in the media of both the plaque free and plaque laden brachiocephalic artery.

FIG. 9. Biodistribution of K-(DOTA-Gd)-YPDHVQYTHY showing renal clearance and preferential uptake in brachiocephalic artery.

### DETAILED DESCRIPTION

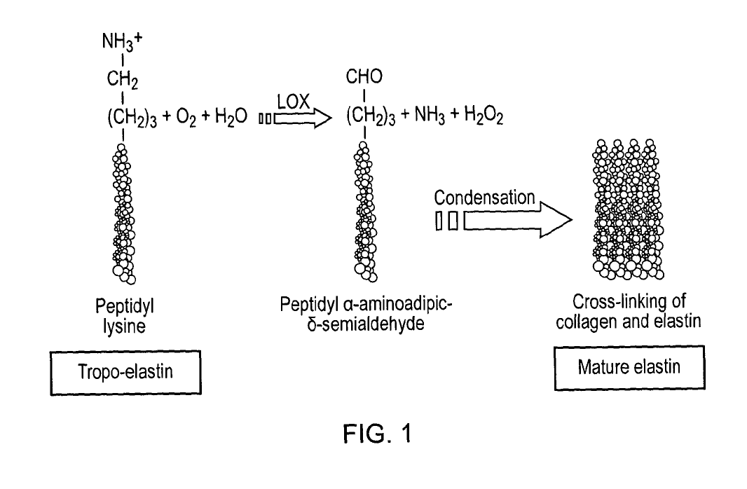

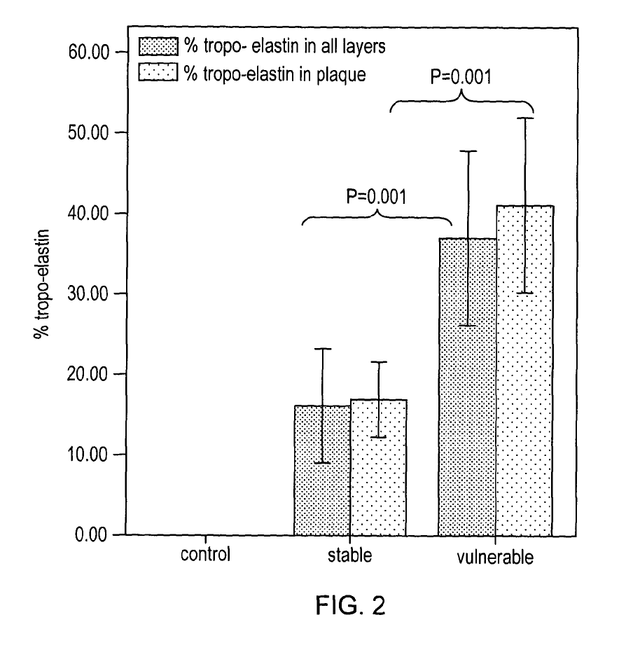

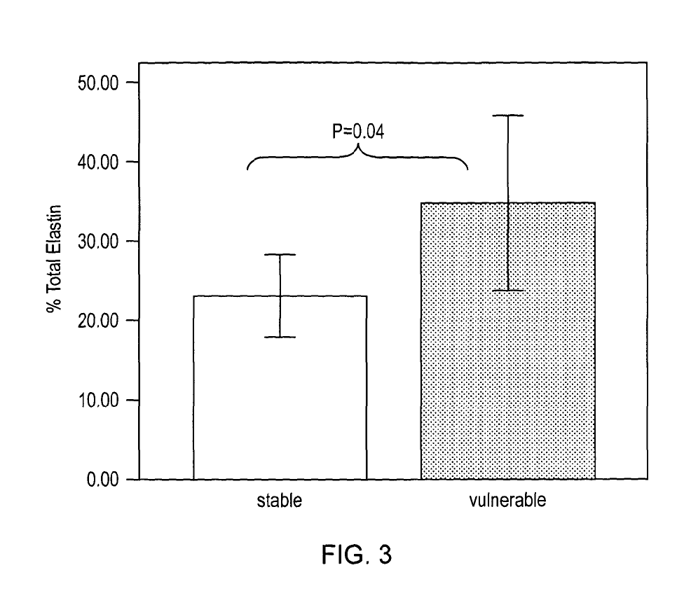

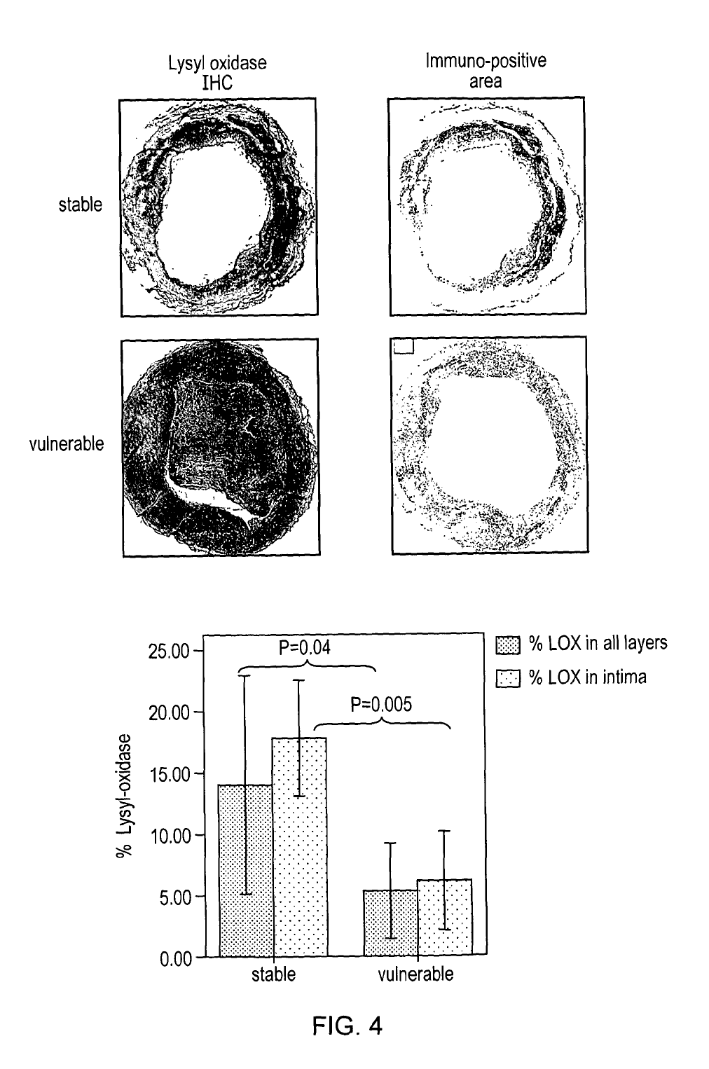

Tropoelastin-specific or lysyl oxidase-specific binding agent Tropoelastin is a matrix protein, which is synthesized to form part of the walls of blood vessels. After expression of immature tropoelastin, it is covalently cross-linked by the enzyme lysyl-oxidase (LOX) to structural mature elastin (FIG. 1), which provides tensile strength to the vessel wall. The present invention is therefore concerned with conjugates that are capable of differentiating between de novo synthesized tropoelastin and mature cross-linked elastin, especially in vivo, the former being associated with an increased risk of plaque instability and rupture, leading to AMI and/or stroke and/or aortic aneurysm. The sequence of human tropoelastin, lysyl oxidase, and elastin are available on sequence databases along with the sequences of the corresponding polypeptides in animal models such as rabbits (see also Sequences section below). Tropoelastin from other species may also be used to design specific binding peptides or for screening antibody based binding agents. It may be advantageous to design peptides or antibodies that are capable of specifically binding to tropoelastin of more than one species, for example to enable the same conjugate to be used for imaging plaques in an animal model and in human patients.

In some cases, in accordance with any one of the aspects of the present invention, the tropoelastin-specific binding peptide is capable of specifically binding tropoelastin. In some cases, in accordance with any one of the aspects of the present invention, the tropoelastin-specific binding peptide substantially does not bind to elastin. In a preferred embodiment, the tropoelastin-specific binding agent is capable of specifically binding tropoelastin in vivo and substantially does not bind to elastin in vivo.

In some cases, in accordance with any one of the aspects of the present invention, the tropoelastin-specific binding peptide is specific for tropoelastin as compared to other intravascular components or proteins. In a preferred embodiment, the tropoelastin-specific binding agent is specific for tropoelastin as compared to other intravascular components or proteins in vivo.

Generally, the tropoelastin-specific binding agent may be a polypeptide or peptide that is capable of specifically binding to tropoelastin or may be an antibody molecule capable of specifically binding to tropoelastin. In a preferred embodiment, the tropoelastin-specific binding agent may be a polypeptide or peptide that is capable of specifically binding to tropoelastin in vivo or may be an antibody molecule capable of specifically binding to tropoelastin in vivo. Equally, the lysyl-oxidase-specific binding agent may be a polypeptide or peptide that is capable of specifically binding to lysyl oxidase or may be an antibody molecule capable of specifically binding to lysyl oxidase.

Examples of tropoelastin-specific binding peptides include peptides having the amino acid sequence VVGSPSAQDEASPLS, EGFEPG or YPDHVQYTHY. In some cases, in accordance with any one of the aspects of the present invention, the tropoelastin-specific binding peptide consists of the sequence VVGSPSAQDEASPLS, EGFEPG or YPDHVQYTHY. The skilled person could readily design alternative peptide sequences using the known amino acid sequences of polypeptides known to bind to tropoelastin and/or lysyl oxidase, taking account of the need to avoid cross-reaction, for example, in the case of tropoelastin-specific binding agents, not to bind to a significant extent to mature elastin, especially in vivo. In the examples, the peptides used were chemically synthesized by Peptide Synthetics (Peptide Protein Research Ltd) after they had been designed.

In some cases, in accordance with any one of the aspects of the present invention, the tropoelastin-specific binding peptide comprises a sequence of at least 4, 6, 8, 10, 12 or 14 amino acids from the amino acid sequence VVGSPSAQDEASPLS. In some cases, in accordance with any one of the aspects of the present invention, the tropoelastin-specific binding peptide is not more than 50, not more than 30, 20, 18, or 16 amino acids in length. In some cases, in accordance with any one of the aspects of the present invention, the tropoelastin-specific binding peptide comprises or consists of the amino acid sequence VVGSPSAQDEASPLS. In some cases, in accordance with any one of the aspects of the present invention, the tropoelastin-specific binding peptide comprises a sequence of at least 4, 6 or 8 amino acids from the amino acid sequence YPDHVQYTHY. In some cases, in accordance with any one of the aspects of the present invention, the tropoelastin-specific binding peptide is not more than 50, not more than 30, 20, 18, 16, 14, 12 or 10 amino acids in length. In some cases, in accordance with any one of the aspects of the present invention, the tropoelastin-specific binding peptide comprises or consists of the amino acid sequence YPDHVQYTHY.

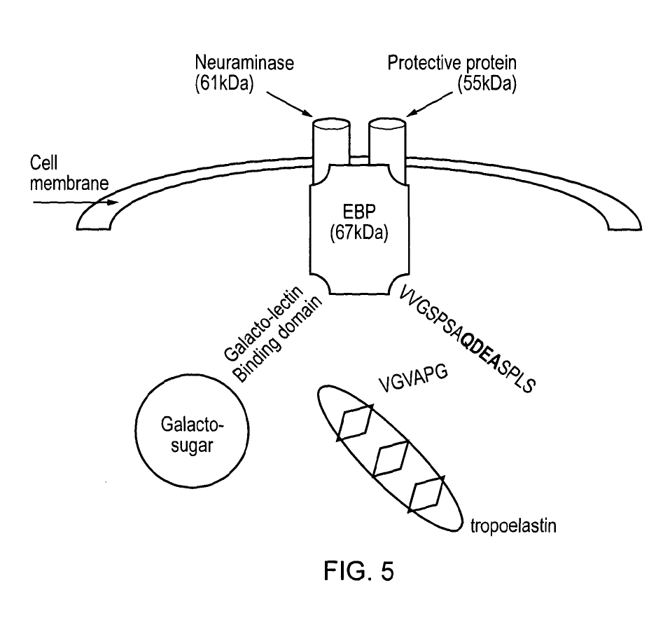

In the present invention, the tropoelastin-specific binding agent may be a peptide or an antibody molecule capable of binding amino acid sequence VGVAPG. In some cases, in accordance with any one of the aspects of the present invention, the tropoelastin-specific binding agent may be a peptide comprising the amino acid sequence QDEA. In some cases, in accordance with any one of the aspects of the present invention, the tropoelastin-specific binding peptide is not more than 50, not more than 30, 20, 18, 16, 14, 12, or 10 amino acids in length. Without wishing to be bound by any particular theory, the amino acid residues QDEA on the tropoelastin-specific binding agent are thought to bind the tropoelastin hexapeptide VGVAPG (FIG. 5).

In the present invention, the tropoelastin-specific binding agent may be a peptide or an antibody molecule capable of specifically binding to tropoelastin, and preferably does not substantially bind to elastin and/or other components of the vascular system. In a preferred embodiment, the tropoelastin-specific binding agent may be a peptide or an antibody molecule capable of specifically binding to tropoelastin, and preferably capable of not substantially binding to elastin and/or other components of the vascular system in vivo. The tropoelastin-specific binding agent (e.g. a peptide or an antibody molecule) may have a dissociation constant for tropoelastin of less than 50 nM, less than 40 nM, less than 30 nM, less than 20 nM, less than 10 nM, or less than 1 nM. In contrast, preferably the tropoelastin-specific binding agent (such as an anti-tropoelastin antibody or peptide) may have a dissociation constant for elastin of more than 100 μmol/L. For example, the tropoelastin-specific binding agent (such as an anti-tropoelastin antibody or peptide) may have a dissociation constant for in vivo elastin (e.g. elastin present in or derived from a mammalian, e.g. human, subject) of more than 1, 10, 100 or 200 μmol/L.

In the present invention, where the lysyl oxidase-specific binding agent is a peptide or an antibody molecule capable of specifically binding to lysyl oxidase, and not to other components of the vascular system, the peptide or anti-lysyl oxidase antibody may have a dissociation constant for lysyl oxidase of less than 50 nM, less than 40 nM, less than 30 nM, less than 20 nM, less than 10 nM, or less than 1 nM.

Binding kinetics and affinity (expressed as the equilibrium dissociation constant K_{d}) of the tropoelastin specific peptide or anti-tropoelastin antibody molecules may be determined using standard techniques, such as surface plasmon resonance e.g. using BIAcore analysis.

An anti-tropoelastin antibody molecule or anti-lysyl oxidase antibody molecules as described herein may be an immunoglobulin or fragment thereof, and may be natural or partly or wholly synthetically produced, for example a recombinant molecule. One example of an anti-tropoelastin antibody molecule can be purchased from Calbiochem Cat No. 324756.

Anti-tropoelastin antibody molecules or anti-lysyl oxidase antibody molecules may include any polypeptide or protein comprising an antibody antigen-binding site, including Fab, Fab₂, Fab₃, diabodies, triabodies, tetrabodies, minibodies and single-domain antibodies, as well as whole antibodies of any isotype or sub-class. Antibody molecules and methods for their construction and use are described, in for example Holliger & Hudson, Nature Biotechnology 23(9):1126-1136 (2005).

In some preferred embodiments, the anti-tropoelastin antibody molecule or anti-lysyl oxidase antibody molecules may be a whole antibody. For example an IgG, IgA, IgE or IgM or any of the isotype sub-classes, particularly IgG1 and IgG4. The anti-tropoelastin antibody molecules may be monoclonal antibodies.

Anti-tropoelastin antibody molecules or anti-lysyl oxidase antibody molecules may be chimeric, humanised or human antibodies.

Anti-tropoelastin antibody molecules or anti-lysyl oxidase antibody molecules as described herein may be isolated, in the sense of being free from contaminants, such as antibodies able to bind other polypeptides and/or serum components. Monoclonal antibodies are preferred for some purposes, though polyclonal antibodies may also be employed.

Anti-tropoelastin antibody molecules or anti-lysyl oxidase antibody molecules may be obtained using techniques, which are standard in the art. Methods of producing antibodies include immunising a mammal (e.g. mouse, rat, rabbit, horse, goat, sheep or monkey) with the protein or a fragment thereof. Antibodies may be obtained from immunised animals using any of a variety of techniques known in the art, and screened, preferably using binding of antibody to antigen of interest. For instance, Western blotting techniques or immunoprecipitation may be used (Armitage et al., 1992, Nature 357: 80-82). Isolation of antibodies and/or antibody-producing cells from an animal may be accompanied by a step of sacrificing the animal.

As an alternative or supplement to immunising a mammal with a peptide, an antibody specific for a protein may be obtained from a recombinantly produced library of expressed immunoglobulin variable domains, e.g. using lambda bacteriophage or filamentous bacteriophage which display functional immunoglobulin binding domains on their surfaces; for instance see WO92/01047. The library may be naive, that is constructed from sequences obtained from an organism, which has not been immunised with any of the proteins (or fragments), or may be one constructed using sequences obtained from an organism, which has been exposed to the antigen of interest.

In some embodiments, anti-tropoelastin antibody molecules or anti-lysyl oxidase antibody molecules may be produced by any convenient means, for example a method described above, and then screened for differential binding to tropoelastin relative to elastin (and/or another component of the vessel wall). Suitable screening methods are well-known in the art and enable those skilled in the art to identify an antibody which displays increased binding to tropoelastin, relative to non-tropoelastin proteins such as elastin, or antibodies capable of binding to lysyl oxidase.

After production and/or isolation, the biological activity of an anti-tropoelastin antibody molecule or anti-lysyl oxidase antibody molecules may be tested, for example using the binding experiments described above or in the production of a conjugate so that its properties as an imaging agent may be determined.

Antibody molecules normally comprise an antigen-binding domain comprising an immunoglobulin heavy chain variable domain (VH) and an immunoglobulin light chain variable domain (VL), although antigen binding domains comprising only a heavy chain variable domain (VH) are also possible (e.g. camelid or shark antibodies).

The term also covers any polypeptide or protein comprising an antibody-binding domain. Antibody fragments which comprise an antigen binding domain are such as Fab, scFv, Fv, dAb, Fd; and diabodies. It is possible to take monoclonal and other antibodies and use techniques of recombinant DNA technology to produce other antibodies or chimeric molecules, which retain the specificity of the original antibody. Such techniques may involve introducing DNA encoding in the immunoglobulin variable region, or the complementarity determining regions (CDRs), of an antibody to the constant regions, or constant regions plus framework regions, of a different immunoglobulin. See, for instance, EP 0 184 187 A, GB 2,188,638 A or EP 0 239 400 A.

Tropoelastin-specific antibodies and anti-lysyl oxidase antibody molecules are known in the art and are commercially available from sources such as Calbiochem/Abcam. Alternatively, the skilled person could readily produce and screen candidate antibodies as discussed above.

### C: Imaging Probes

In addition to the tropoelastin-specific binding agent, the conjugates of the present invention include an imaging probe capable of detection by an imaging technique such as MRI, PET or SPECT, or combinations thereof. Examples of types of imaging probe include radionuclides, optical labels or paramagnetic labels. The present invention may also involve the use of further labelled probes that may be linked to or associated with the conjugates, for example to enable multi-modal imaging to be carried out. The possibility to incorporate optical probes as well as radionuclides and MRI contrast agents provides the opportunity to combine modalities to enhance diagnosis and detection, for example the location of disease at the whole body level can be identified by whole body scanning with PET or SPECT. Similarly, combined PET and MR imaging can provide the advantage of high sensitivity (PET, SPET), quantification of signal (PET) and anatomical resolution (MR), and measurement of the microenvironment (MR contrast enhancement).

One preferred class of conjugates of the present invention are MRI agents that comprise a tropoelastin specific binding agent linked to a group capable of complexation to a MRI active atom such as gadolinium. An alternative MRI signal element may include iron oxides. A further possibility is the use of ¹⁹F as a NMR or MRI label and/or ¹⁸F as a label, e.g. for PET or CT imaging.

In one embodiment, the group capable of complexation to a MRI active atom comprises DOTA. In some embodiments the group capable of complexation to a MRI active atom is DOTA-lysine.

The radionuclide probes used in accordance with the present invention may use a range of different radionuclides depending on the application for which the probes are intended. Examples of radionuclides that may form part of the probes of the present invention include technetium, rhenium, copper, cobalt, gallium, yttrium, lutetium, indium, zirconium, carbon, iodine, fluorine and astatine isotopes such as Tc-99m, Ga-67, In-111, I-123 (SPECT), Cu-64, Cu-60, Cu-61, Cu-62, Tc-94m, Ga-68, Co-55, F-18, C-11, I-124, Zr-89 (PET), Cu-67, Re-186, Re-188, Y-90, Lu-177, I-131 (radionuclide therapy). The present invention may employ the radionuclides alone or in combinations. In general, technetium isotopes are employed for imaging purposes, rhenium isotopes for therapeutic purposes and copper and halogen isotopes for both imaging and therapy.

Examples of optical probes include fluorophores such as fluorescein, luminescent molecules and complexes such as lanthanide complexes.

### Linkers and Conjugation Chemistry

In some embodiments, the conjugates may comprise a linker or functional group to join the tropoelastin-specific binding agent and the imaging probe. The linker may be a short peptide sequence or may be a chemical linker. The use of peptide linker sequences will be between 6 and 25 amino acids in length, more preferably between 9 and 16 amino acids in length is known in the art. Linked typically comprise reactive groups for linking to the binding agent and imaging probe, such as a free cysteine residue.

### Conjugates

In some embodiments, the conjugate is one of:

(DOTA-Gd)-VVGSPSAQDEASPLS,
(DOTA-Gd)-VVGSPSAQDEASPLS-K(DOTA-Gd),
K(DOTA-Gd)-VVGSPSAQDEASPLS-K(DOTA-Gd),
K(DOTA-Gd)K(DOTA-Gd)-VVGSPSAQDEASPLS,
K(DOTA-Gd)-VVGSPSAQDEASPLS,
K(DOTA-Gd)-YPDHVQYTHY-K(DOTA-Gd),
(DOTA-Gd)-YPDHVQYTHY-K(DOTA-Gd),
(DOTA-Gd)-YPDHVQYTHY,
K(DOTA-Gd)-YPDHVQYTHY
or
K(DOTA-Gd)K(DOTA-Gd)-YPDHVQYTHY.

### Uses

In one aspect, the present invention provides conjugates for use in methods of imaging tropoelastin in the cardiovascular system of a subject, and in particular for imaging plaques. The method generally entails the steps of:
(a) administering to the subject a composition comprising a conjugate for imaging cardiovascular plaques comprising a tropoelastin-specific binding agent and an imaging probe;
(b) allowing the imaging agent to bind to any tropoelastin present in plaques in the vascular system of the subject;
(c) detecting the imaging probe to determine the presence of the plaques.

In order to come into contact with and bind tropoelastin in plaques, generally a composition comprising the conjugates will be for intravenous administration to the subject. After a suitable delay for binding to take place, the imaging probe may be detected using an imaging technique as described herein. The results of the detecting step may then be used to quantify the tropoelastin present in plaques, and may then be used to assess plaque burden and/or the likelihood of plaque rupture and/or monitor disease progression and/or response to therapy. The aim of this would be to determine a prognosis for a subject, in particular as regards the risk of having AMI, a stroke and/or an aortic aneurysm, and/or to help determine therapeutic interventions intended to improve the condition of the subject.

Although the primary means of imaging using the conjugates employs MRI, this may be used in conjunction with other nuclear medicine imaging techniques, such as Single Photon Emission Tomography (SPET), an imaging technique that detects gamma rays emitted from a radionuclide to produce a three dimensional image of the distribution of the radionuclide in a sample or subject, and Positron Emission Tomography (PET), an imaging technique that provides three-dimensional images by detecting pairs of gamma rays emitted indirectly by a positron-emitting radionuclide introduced into a sample or subject. By way of example SPET studies can be carried out using ^{99m}Tc and PET studies using ^{94m}Tc. The skilled person, however, will be aware of other suitable SPET and PET radionuclides that can be employed in the present invention. Generally, the present invention may be employed for positron emission tomography (PET), single photon emission tomography (SPET), optical (OI) and/or magnetic resonance imaging (MRI) by appropriate selection of imaging probe.

Thus, the conjugates of the present invention may be used in methods of multi-modal imaging, that is where information or images are derived from two different techniques, either by the detection of the imaging probe capable of detection using two different techniques or by providing a second label at the site in the biological system where the nanoparticles become localised, most conveniently by linking or associating the second label with the conjugates as explained in detail above. Multi-modal studies will be co-registered and may entail simultaneous imaging with two modalities or may need to take place in two steps, but generally employ the same sample so that spatial information obtained using the two techniques can be compared. Examples of multi-modal imaging include PET/CT, SPET/CT, PET/MR and SPET/MR.

By way of example, the following exemplary protocol may be used imaging according to the methods of the present invention. For visualization of contrast agent uptake in the coronary artery walls and large vessels such as the aorta, a navigator-gated, cardiac-triggered, fat-suppressed T1-weighted 3D gradient echo inversion recovery targeted or whole heart sequence (3D IR TFE or 3D IR SSFP) may be used. Imaging parameters of a 3D IR TFE sequence may include field of view=320×320 mm, matrix=256×256, acquired in-plane resolution=1.25×1.25 mm, reconstructed slice thickness=1.5 mm (acquired: 3 mm), acquisition window=80 to 100 ms, repetition time/echo time=5.8 ms/1.9 ms, flip angle=30°, startup cycles=5, and number of slices=20 but may differ for the whole heart and SSFP protocol. The patient-specific inversion time (TI) will be adjusted to null blood signal of blood using a Look Locker sequence.

### Materials and Methods

### Tropoelastin-Specific Binding Agents

Three different peptides (VVGSPSAQDEASPLS, EGFEPG and YPDHVQYTHY) were chosen for the tropoelastin-binding agent and conjugated with DOTA-lysine for gadolinium and PET/SPECT labelling.

### Experimental Design

The proof of principle experiments described herein for the in vivo and ex vivo testing of the conjugates used mouse and rabbit models.

### Binding Studies

Binding studies with tropoelastin and TNF-alpha coated petri dishes will be performed to demonstrate specificity of the agents. Furthermore, transmission electron microscopy of vessel specimens will be performed for elastin and macrophage visualization while X-ray spectra will be acquired for colocalization with gadolinium distribution in plaque laden vessel wall samples.

### Histology

Animals will be euthanized immediately after MRI. Subsequently, the brachiocephalic artery and abdominal aorta will be excised and cut into 3 mm segments. Sections will be cut into 3 μm slices for paraffin-embedded and 6 μm for OCT-embedded sections. Sections will be then stained with hematoxylin and eosin (H&E) for cellular infiltration, Miller's elastica van Gieson (EVG) for elastin and Masson's trichrome, and Picrosirius Red for plaque morphology and collagen deposition. In addition, immunostaining with specific antibodies for tropoelastin, TNF-alpha and LOX will be performed. Mass spectroscopy (MS) will be applied to quantify the molar concentration of Gd in the investigated vessel specimens.

### ApoE Mouse Model

MRI will be performed in a mouse model of progressive atherosclerosis at 4, 8 and 12 weeks post commencement of a high fat diet and in a model of angiotensin-II (Ang-II) induced aortic aneurysm formation at 1, 2, 3 and 4 weeks post Ang-II releasing mini pump implantation. Ten mice will be scanned at each time point either receiving the tropoelastin or TNF-alpha binding contrast agent (CA) resulting in a total of 60 and 80 mice, respectively. Animals will undergo a pre and post contrast MRI session at each time point and subsequently will be sacrificed for validation with histology, immunostaining, electron and mass spectroscopy. To demonstrate treatment effects, 10 mice will be scanned after 12 weeks of therapy with statins with the tropoelastin binding CA. To demonstrate the role of LOX in tropoelastin synthesis, 10 mice will be scanned with the tropoelastin CA 12 weeks after commencement of LOX inhibitor treatment.

### Plaque Rupture Model

New Zealand White rabbits will be fed a high cholesterol diet (Special Diets Services) for 2 weeks and then undergo balloon injury of the abdominal aorta. Subsequently, the high fat diet will be continued for another 6 weeks followed by 4 weeks of normal diet. Plaques using this protocol have been shown to develop similar features compared with AHA type II-VI lesions (excluding the presence of calcified lesions). MRI will be performed with the tropoelastin binding MR contrast agent prior to triggering of plaque rupture using histamine and Russel's viper venom (RVV). 48 h after induction of plaque rupture/erosion, MRI will be repeated in order to detect the presence of intraluminal thrombi and to correlate thrombus location with pre-trigger tropoelastin-Gd. A total of 16 rabbits will be scanned resulting in approximately 8 (50%) rabbits with and without plaque rupture. Immediately after the last scan, animals will be sacrificed for validation with histology, immunostaining, mass and electron spectroscopy.

### EXAMPLES

Rabbit aortic segments were cryo-protected (30% sucrose), embedded in tissue freezing medium and stored at −80° C. Serial 10 μm thick cross-sections (spanning 300 μm length) were collected with 500 μm intervals. Sections were used for Masson's trichrome for the detection the general plaque morphology, Van Gienson elastin staining for the detection of mature and immature elastin fibers and immunohistochemistry for the detection of tropoelastin fibers, LOX, and macrophages. Disrupted plaques were classified using the Masson's trichrome staining and included both ruptured and eroded, as defined for human plaques. Non-disrupted plaques included those without an overlying thrombus.

Immunohistochemistry was performed by the avidin-biotin-peroxidase method (Vector Laboratories, No. PK-6102). Anti-rabbit polyclonal antibodies for tropoelastin (Calbiochem, #324756), LOX (IMGENEX, #IMG-6442A) and macrophages (Dako, clone RAM11, No. M0633) were used and the following steps were followed: 1) sections were incubated in 10% formalin for 20 minutes at room temperature to adhere the tissue sections on the slides; 2) sections were incubated in a citrate-based solution (10 mM citric acid, 0.05% Tween 20, pH 6.0) (Vector Laboratories, Burlingame, Calif., No. H-3300) at 100° C. for 20 min using a pressure cooker to retrieve the epitope; 3) 1% hydrogen peroxide for 10 min at room temperature to block endogenous peroxidase activity; 4) 10% horse serum for 60 min to reduce nonspecific binding of the antiserum; 5) primary antibodies for 2 h at room temperature. Negative control sections were incubated with 10% horse sera only; 6) biotinylated horse anti-mouse immunglobulin G (at a dilution of 1:200) for 1 hr at room temperature; and 7) avidin-Elite biotinylated horseradish peroxidase complex (Vectastain^{Elite}, Vector Laboratories, No. PK-6102) at a dilution of 1:50 for 1 hr at room temperature. Immunoreactive sites were visualized by incubation with 3,3-diaminobenzidine (DAB substrate chromogen, Vector Laboratories, No. SK-4100) at a dilution of 1:50 for 3-5 min. Tris buffered saline (pH 7.4) was used to dilute each solution and to wash the sections three times between each step. Finally, tissue sections were counterstained with hematoxylin (1 min).

Using an antibody that appears to bind to the immature (tropo) elastin and a rabbit model of controlled plaque disruption we found that:

1. There is increase deposition of tropoelastin fibers during the progression of atherosclerosis as well as in vulnerable plaque.
2. In the initial steps the tropoelastin fibers are scattered throughout the intima and in the later stages they increase in density and they are also found in the adventitia.
3. The increase elastin content in vulnerable plaque may be used in molecular imaging for the in vivo detection of such lesions.
4. In some cases, the tropoelastin fibers appear to co-localize with CD68-positive macrophages indicating that macrophages maybe a source of elastin.
5. However, there are also cases in which the macrophages do not co-localize with elastin fibers indicating that there might be a diversity of macrophage sub-populations with different local functionality.

Further experiments investigated imaging using tropoelastin-specific binding peptides.

Potential cleavage sites of the peptides VVGSPSAQDEASPLS and YPDHVQYTHY were investigated. Only enzymes that are primarily present in the digestive system were found to cleave the peptides VVGSPSAQDEASPLS and YPDHVQYTHY. None of these enzymes were reported in blood or plaques and thus are unlikely to cleave the peptide VVGSPSAQDEASPLS or YPDHVQYTHY before it binds to the vessel wall/plaque specific target, tropoelastin.

A protein BLAST was performed to screen for homologies. The amino acid sequences VVGSPSAQDEASPLS and YPDHVQYTHYK were only found in proteins described to interact with tropoelastin (elastin-binding protein (EBP) and Microfibril-associated Glycoprotein-1 (MAGP-1) respectively) and not in other proteins. These results suggest that the chosen peptides are highly specific for the protein of interest, tropoelastin.

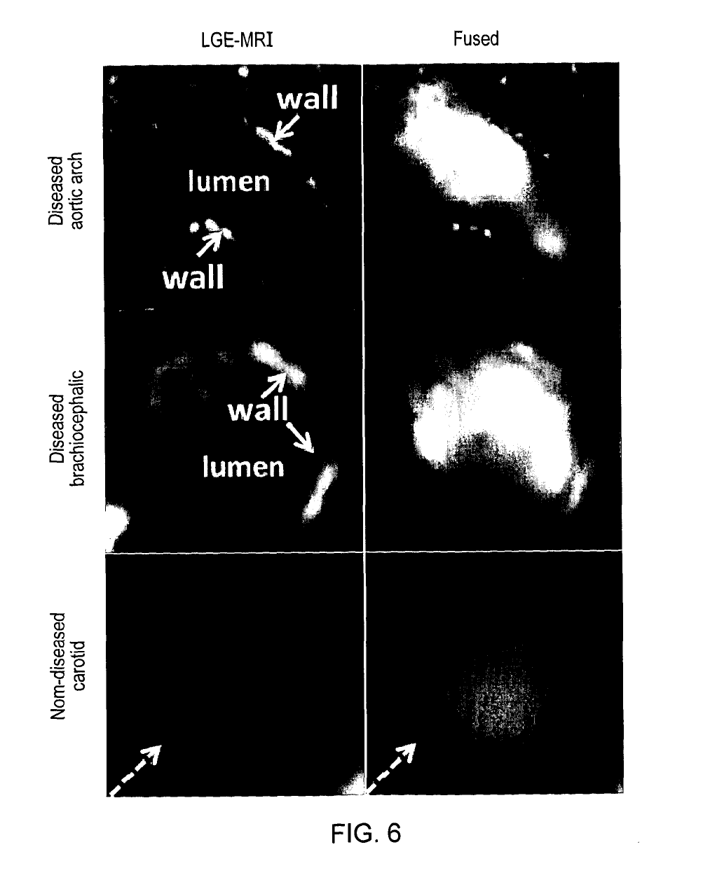

In-vivo experiments in 12 weeks high-fat diet (HFD) fed ApoE^{-/-} mice injected with gadolinium labelled (DOTA-Gd)-VVGSPSAQDEASPLS showed a favourable biodistribution with preferential uptake in the plaque-laden brachiocephalic artery (BCA) and aortic arch but not in the plaque-free carotid artery (FIG. 6), and rapid renal clearance allowing for imaging as early as 1 hour post contrast injection.

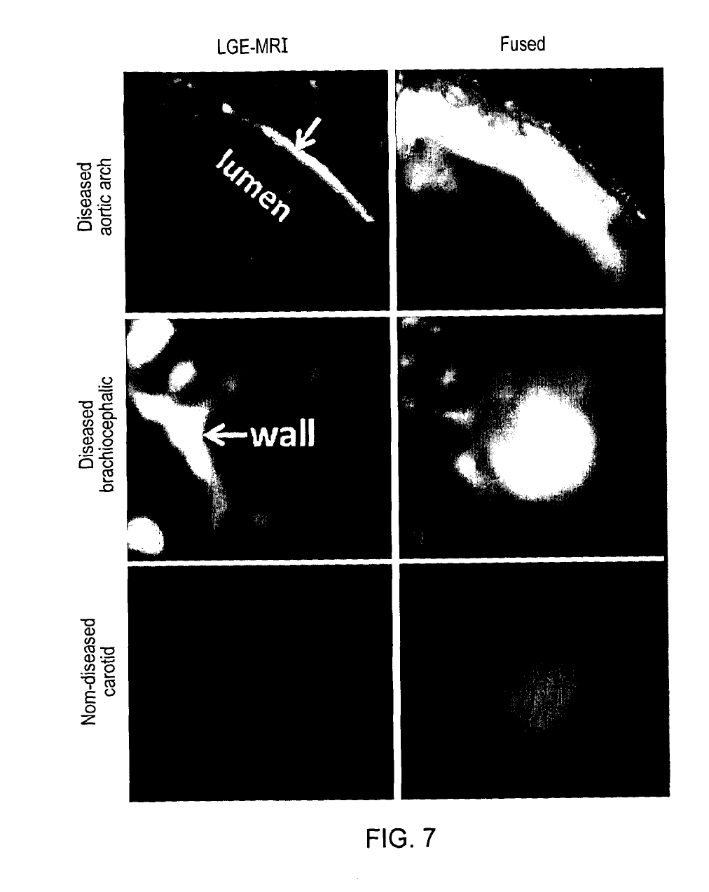

In-vivo experiments in HFD fed ApoE^{-/-} mice with gadolinium-labelled K-(DOTA-Gd)-YPDHVQYTHY showed promising results with uptake in the plaque laden brachiocephalic and aortic arch and no uptake in plaque free carotid artery (FIG. 7). The peptide also showed favorable biodistribution with rapid renal clearance and preferential uptake in the BCA (FIG. 9).

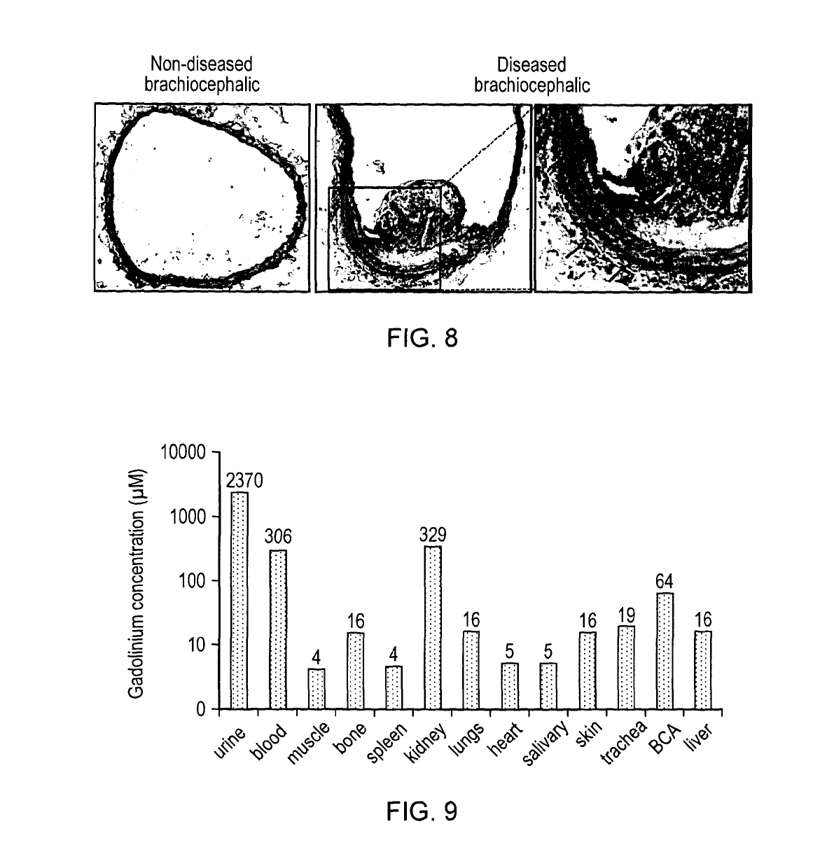

Immunohistochemistry verified the presence of tropoelastin in the neointima and adventitia of the diseased BCA, and the absence of tropoelastin in the media of both the plaque-laden (diseased) and plaque-free (non-diseased) BCA vessel walls (FIG. 8).

The bound relaxivity at 3T was measured as 20.88 mM⁻¹ s⁻¹.

All documents mentioned in this specification are incorporated herein by reference in their entirety.

### Sequences

| 1. Tropoelastin-specific binding peptides | |
|---|---|
| VVGSPSAQDEASPLS | |
| EGFEPG | |
| YPDHVQYTHY | |
| 2. Human tropoelastin | |
| 1 | magltaaapr pgvlllllsi lhpsrpggvp gaipggvpgg vfypgaglga lgggalgpgg |
| 61 | kplkpvpggl agaglgaglg afpavtfpga lvpggvadaa aaykaakaga glggvpgvgg |
| 121 | lgvsagavvp qpgagvkpgk vpgvglpgvy pggvlpgarf pgvgvlpgvp tgagvkpkap |
| 181 | gvggafagip gvgpfggpqp gvplgypika pklpggyglp yttgklpygy gpggvagaag |
| 241 | kagyptgtgv gpqaaaaaaa kaaakfgaga agvlpgvgga gvpgvpgaip giggiagvgt |
| 301 | paaaaaaaaa akaakygaaa glvpggpgfg pgvvgvpgag vpgvgvpgag ipvvpgagip |
| 361 | gaavpgvvsp eaaakaaaka akygarpgvg vggiptygvg aggfpgfgvg vggipgvagv |
| 421 | pgvggvpgvg gvpgvgispe aqaaaaakaa kygaagagvl gglvpgpqaa vpgvpgtggv |
| 481 | pgvgtpaaaa akaaakaaqf glvpgvgvap gvgvapgvgv apgvglapgv gvapgvgvap |
| 541 | gvgvapgigp ggvaaaaksa akvaakaqlr aaaglgagip glgvgvgvpg lgvgagvpgl |
| 601 | gvgagvpgfg agadegvrrs lspelregdp sssqhlpstp ssprvpgala aakaakygaa |
| 661 | vpgvlgglga lggvgipggv vgagpaaaaa aakaaakaaq fglvgaaglg glgvgglgvp |
| 721 | gvgglggipp aaaakaakyg aaglggvlgg agqfplggva arpgfglspi fpggaclgka |
| 781 | cgrkrk |
| 3. Mouse tropoelastin | |
| 1 | magltavvpq pgvllillln llhpaqpggv pgavpgglpg gvpggvyypg agigglgggg |
| 61 | galgpggkpp kpgagllgtf gagpgglgga gpgaglgafp agtfpgagal vpggaagaaa |
| 121 | aykaaakaga glggvggvpg gvgvggvpgg vgvggvpggv gvggvpggvg giggigglgv |
| 181 | stgavvpqvg agigaggkpg kvpgvglpgv ypggvlpgtg arfpgvgvlp gvptgtgvka |
| 241 | kapggggafs gipgvgpfgg qqpgvplgyp ikapklpggy glpytngklp ygvagaggka |
| 301 | gyptgtgvgs qaaaaaakaa kygaggagvl pgvggggipg gagaipgigg iagagtpaaa |
| 361 | aaakaaakaa kygaagglvp ggpgvrlpga gipgvggipg vggipgvggp giggpgivgg |
| 421 | pgavspaaaa kaaakaakyg arggvgipty gvgaggfpgy gvgagaglgg aspaaaaaaa |
| 481 | kaakygagga galgglvpga vpgalpgavp avpgaggvpg agtpaaaaaa aaakaaakag |
| 541 | lgpgvggvpg gvgvggipgg vgvggvpggv gpggvtgiga gpgglggags paaaksaaka |
| 601 | aakaqyraaa glgagvpgfg agagvpgfga gagvpgfgag agvpgfgaga gvpgfgagav |
| 661 | pgslaaskaa kygaagglgg pgglggpggl ggpgglggag vpgrvagaap paaaaaaaka |
| 721 | aakaaqyglg gagglgaggl gagglgaggl gagglgaggl gagglgaggl gagggvspaa |
| 781 | aakaakygaa glggvlgarp fpgggvaarp gfglspiypg ggagglgvgg kppkpyggal |
| 841 | galgyqgggc fgkscgrkrk |
| 4. Human lysyl oxidase | |
| 1 | mrfawtvlll gplqlcalvh cappaagqqq ppreppaapg awrqqiqwen ngqvfsllsl |
| 61 | gsqyqpqrrr dpgaavpgaa nasaqqprtp illirdnrta aartrtagss gvtagrprpt |
| 121 | arhwfqagys tsrareagas raenqtapge vpalsnlrpp srvdgmvgdd pynpykysdd |
| 181 | npyynyydty erprpggryr pgygtgyfqy glpdlvadpy yiqastyvqk msmynlrcaa |
| 241 | eenclastay radvrdydhr vllrfpqrvk nqgtsdflps rpryswewhs chqhyhsmde |
| 301 | fshydlldan tqrrvaeghk asfcledtsc dygyhrrfac tahtqglspg cydtygadid |
| 361 | cqwiditdvk pgnyilkvsv npsylvpesd ytnnvvrcdi rytghhayas gctispy |
| 5. Mouse lysyl oxidase | |
| 1 | mrfawavlll gplqlcpllr capqtprepp aapgawrqti qwenngqvfs llslgagyqp |
| 61 | qrrrdpsata rrpdgdaasq prtpilllrd nrtastrart pspsgvaagr prpaarhwfq |
| 121 | agfspsgard gasrraanrt aspqppqlsn lrppshidrm vgddpynpyk ysddnpyyny |
| 181 | ydtyerprpg srnrpgygtg yfqyglpdlv pdpyylqast yvqkmsmynl rcaaeencla |
| 241 | ssayradvrd ydhrvllrfp qrvknqgtsd flpsrprysw ewhschqhyh smdefshydl |
| 301 | ldantqrrva eghkasfcle dtscdygyhr rfactahtqg lspgcydtya adidcqwidi |
| 361 | tdvqpgnyil kvsvnpsylv pesdytnnvv rcdirytghh ayasgctisp y |
| 6. PREDICTED rabbit lysyl oxidase | |
| 1 | mlcswtvlll gplqlcalvc gapqaagqqq ppreppaapg awrqriqwen ngqvfsllsl |
| 61 | gaqyqpqrrr dagaaapgaq raagpqqrtp vlllrdnrta aasrprpagr hwfqagyasp |
| 121 | gardagasra gnrtaqgepp alsnlrppsh vdrmvgddpy npykysddnp yynyydtyer |
| 181 | prpgsryrpg ygtgyfqygl pdlvpdpyyi qastyvqkms mynlrcaaee nclassayra |
| 241 | dvrdydhrvl lrfpqrvknq gtsdflpsrp ryswewhsch qhyhsmdefs hydlldantq |
| 301 | rrvaeghkas fcledtscdy gyhrrfacta htqglspgcy dtyaadidcq widitdvqpg |
| 361 | nyilkvsvnp sylvpesdyt nnvvrcdiry tghhayasgc tisp |

### REFERENCES

1. Krettek et al ‘Elastogenesis in human arterial disease: A role for macrophages in disordered elastin synthesis’ Arterioscl. Throm. Vas. 23 (2003) 582-587
2. Xu et al ‘Hypercholesterolemia superimposed by experimental hypertension induces differential distribution of collagen and elastin’ Arterioscl. Throm. Vas. 20 (2000) 2566-2572
3. Akima et al ‘Soluble Elastin Decreases in the Progress of Atheroma Formation in Human Aorta’ Circ. J. 73 (2009) 2154-2162
4. Kozel et al ‘Elastic fiber formation: a dynamic view of extracellular matrix assembly using timer reporters’ J. Cell. Physiol. 207 (2006) 87-96
5. Starcher et al ‘Antibody raised to AKAAAKAAAKA sequence on tropoelastin recognizes tropoelastin but not mature crosslinked elastin: A new tool in metabolic and structural studies of elastogenesis’ Connect. Tissue Res. 40 (1999) 273-282
6. WO2011/005322
U.S. Pat. No. 5,972,890 A
8. U.S. Pat. No. 4,877,599 A

1-41. (canceled)

42. A conjugate for imaging plaques comprising a tropoelastin-specific binding agent linked to an imaging probe, wherein the imaging of plaques with said conjugate is for determining the risk of a patient developing a condition caused by plaque rupture or instability, wherein said tropoelastin-specific binding agent is a peptide that comprises a sequence of at least 4 amino acids from an amino acid sequence selected from the group consisting of YPDHVQYTHY (SEQ ID NO: 5) and VVGSPSAQDEASPLS (SEQ ID NO: 1).

43. The conjugate of claim 42, wherein said peptide comprises the amino acid sequence QDEA (SEQ ID NO: 6).

44. The conjugate of claim 42, wherein said tropoelastin-specific binding peptide is capable of binding to amino acid sequence VGVAPG (SEQ ID NO: 2).

45. The conjugate of claim 42, wherein said peptide has an amino acid sequence selected from the group consisting of VVGSPSAQDEASPLS (SEQ ID NO: 1) and YPDHVQYTHY (SEQ ID NO: 5).

46. The conjugate of claim 42, wherein said peptide consists of an amino acid sequence selected from the group consisting of VVGSPSAQDEASPLS (SEQ ID NO: 1) and YPDHVQYTHY (SEQ ID NO: 5).

47. The conjugate of claim 42, wherein said tropoelastin-specific binding agent is specific for human tropoelastin compared to human elastin.

48. The conjugate of claim 42, wherein said tropoelastin-specific binding agent is capable of specifically binding tropoelastin in vivo and substantially does not bind to elastin in vivo.

49. The conjugate of claim 42, wherein said tropoelastin-specific binding agent is specific for tropoelastin as compared to other intravascular components or proteins in vivo.

50. The conjugate of claim 42, wherein said plaques are cardiovascular plaques.

51. The conjugate of claim 50, wherein said cardiovascular plaques are atherosclerotic plaques.

52. The conjugate of claim 42, wherein said condition is selected from the group consisting of acute myocardial infarction (AMI), stroke and aortic aneurysm.

53. The conjugate of claim 42, wherein said imaging of plaques with said conjugate is used to determine a course of treatment for a patient, to assign a patient to a class of patients for a given therapy, to assess plaque burden, to monitor disease progression and/or to determine the response of a patient to a therapy.

54. The conjugate of claim 48, further wherein said tropoelastin-specific binding agent is specific for tropoelastin compared to elastin in an animal model of a condition caused by plaques.

55. The conjugate of claim 42, wherein said imaging probe is for use in an imaging technique selected from the group consisting of MRI, SPECT and PET imaging.

56. The conjugate of claim 42, wherein said imaging probe comprises a label selected from the group consisting of an MRI agent linked to a group capable of complexation of gadolinium; a DOTA-lysine for gadolinium based imaging; a DOTA-lysine for gadolinium based imaging or iron oxide; a radionuclide which is a fluorine, technetium, rhenium, copper, cobalt, gallium, yttrium, lutetium, indium, zirconium, carbon, iodine, fluorine or astatine isotope; an optical label with fluorescent or luminescent properties; and a paramagnetic probe for use as a MRI contrast agent.

57. The conjugate of claim 42, wherein said conjugate is selected from the group consisting of:
(SEQ ID NO: 1)
(DOTA-Gd)-VVGSPSAQDEASPLS,
(SEQ ID NO: 7)
(DOTA-Gd)-VVGSPSAQDEASPLS-K(DOTA-Gd),
(SEQ ID NO: 8)
K(DOTA-Gd)-VVGSPSAQDEASPLS-K(DOTA-Gd),
(SEQ ID NO: 10)
K(DOTA-Gd)K(DOTA-Gd)-VVGSPSAQDEASPLS,
(SEQ ID NO: 9)
K(DOTA-Gd)-VVGSPSAQDEASPLS,
(SEQ ID NO: 11)
K(DOTA-Gd)-YPDHVQYTHY-K(DOTA-Gd),
(SEQ ID NO: 12)
(DOTA-Gd)-YPDHVQYTHY-K(DOTA-Gd),
(SEQ ID NO: 5)
(DOTA-Gd)-YPDHVQYTHY,
(SEQ ID NO: 13)
K(DOTA-Gd)-YPDHVQYTHY,
and
(SEQ ID NO: 3)
K(DOTA-Gd)K(DOTA-Gd)-YPDHVQYTHY.

58. A composition comprising a conjugate according claim 42.

59. A conjugate for use in a method of imaging plaques using a conjugate comprising a tropoelastin-specific binding agent linked to an imaging probe according to claim 1, wherein the imaging of plaques with said conjugate is for determining a risk of a patient developing a condition caused by plaque rupture or instability, the method comprising:
(a) administering to said patient a composition comprising said conjugate;
(b) allowing said conjugate to bind to tropoelastin present in plaques in the vascular system of said patient;
(c) detecting said imaging probe to determine the presence of the plaques; and
(d) determining the risk of said patient developing a condition caused by plaque rupture or instability by the imaging of cardiovascular plaques with said conjugate.

60. A method of imaging plaques using a conjugate comprising a tropoelastin-specific binding agent linked to an imaging probe according to claim 1, wherein the imaging of plaques with said conjugate is for determining a risk of a patient developing a condition caused by plaque rupture or instability, the method comprising:
(a) administering to said patient a composition comprising said conjugate;
(b) allowing said conjugate to bind to tropoelastin present in plaques in the vascular system of said patient;
(c) detecting said imaging probe to determine the presence of the plaques; and
(d) determining the risk of said patient developing a condition caused by plaque rupture or instability by the imaging of cardiovascular plaques with said conjugate.

61. The method of claim 60, wherein said condition is selected from the group consisting of acute myocardial infarction (AMI), stroke and aortic aneurysm.

62. The method of claim 60, further comprising using the imaging of the cardiovascular plaques with said conjugate for (i) determining a course of treatment for a patient; and/or (ii) assigning a patient to a class of patients for a given therapy; and/or (iii) assessing plaque burden, and/or (iv) monitoring disease progression and/or (v) determining the response of a patient to a therapy.

63. The method of claim 60, wherein:
(i) step (c) comprises quantifying the tropoelastin present in plaques; and/or
(ii) said composition is for intravenous administration to the patient; and/or
(iii) the cardiovascular plaques are atherosclerotic plaques.
